(19) United States
(12) Patent Application Publication
(10) Pub. No.: US 2019/0076277 A1
(43) Pub. Date: Mar. 14, 2019

(54) BRAIDED MEDICAL IMPLANT

(71) Applicant: Lombard Medical Limited, Oxfordshire (GB)

(72) Inventors: Peter Phillips, Oxfordshire (GB); Garreth Boland-Tan, Oxfordshire (GB)

(21) Appl. No.: 15/765,606

(22) PCT Filed: Oct. 10, 2016
(86) PCT No.: PCT/GB2016/053147

§ 371 Date: Apr. 3, 2018

(30) Foreign Application Priority Data
Oct. 8, 2015 (GB) 1517813.0

(51) Int. Cl. A61F 2/90; D04C 1/06

(52) U.S. Cl. CPC A61F 2/90; D04C 1/06; D10B 2509/06; A61F 2230/0034; A61F 2250/0039; A61F 2210/0057

(57) ABSTRACT

A medical implant for implanting in a body lumen, includes multiple filamentary components braided into an elongate structure having a first end and a second end and defining an implant lumen, wherein the cross-section of the elongate structure is non-uniform along its length or wherein the cross-section of the elongate structure is uniform along its length but non-circular, and wherein each filamentary component is substantially equal in length as measured from the first end to the second end of the structure.

The present invention relates to a medical implant for implanting in a body lumen, and in particular to a stent or stent graft in which the stent is formed of braided filamentary components.

An endovascular stent graft is designed to exclude the flow of blood to an aneurysm that has been formed within the wall of the lumen (for example, the aorta). This is achieved by accessing the aneurysm via an artery, usually within the patient's leg, with a system designed to deliver, position and deploy the stent graft so that it bridges and seals off the aneurysm.

Multiple filamentary components can be braided into a stent in which the components are individual filaments terminating at the ends of the stent. Alternatively, a braided stent can be formed from a single filament that is threaded to and fro over the surface of an object where there is a near reversal of direction of the filament at the ends of the structure. It will be appreciated that combinations of these alternative structures can also be formed.

Braided stents (such as those disclosed in US 2011/0130819 A1 in the name of Altura Medical, Inc.) can be transformed in shape between a long and narrow extended configuration and a short and broad contracted configuration to enable in vivo deployment. Other braided stent grafts are disclosed in US 2011/0029063 A1 (MKP Structural Design Associates, Inc.); WO 99/30639 (Biocompatibles Limited); and US 2008/0221670 A1 (Clerc et al.).

It has been surprisingly discovered that a non-uniform braided stent can be formed which can be transformed in shape without the filaments kinking or snagging if the length of each filamentary component from one end of the stent to the other is substantially equal.

In a first aspect of the invention, there is provided medical implant for implanting in a body lumen, including multiple filamentary components (for example formed of a shape memory alloy such as nitinol) braided into an elongate structure having a first end and a second end and defining an implant lumen, wherein the cross-section of the elongate structure is non-uniform along its length or wherein the cross-section of the elongate structure is uniform along its length but non-circular, and wherein each filamentary component is substantially equal in length as measured from the first end to the second end of the structure.

By “equal in length as measured from the first end to the second end of the structure” is meant the length of the filamentary component measured along the component from the point of the component at the first end to the point of the component at the second end of the structure.

The implant may have a first longitudinal section in which the elongate structure is not circular in cross-section and a second longitudinal section in which the elongate structure is circular in cross-section. A preferred stent is a D-shape which can be transformed down to a crimped size and which can pop back up again. In essence, the wires in the complex shape need to have the same overall length when the complex shape has been compressed to a cylinder of narrower diameter. If they have different lengths, the compressed shape will try to knot itself up and will not pack or deploy smoothly.

The way in which the invention can be realized can be described by analogy with aeroplane flight paths across the globe. Because the filamentary elements are disposed on a three-dimensional surface, it is possible for them to be identical in length. Consider the length of a flight path of an aeroplane traveling between, say London and New York. This is not calculated simply by drawing a straight line between London and New York on a two-dimensional map; rather, because the surface of the earth is curved, the shortest flight path actually appears as a continuous curve when represented on a two-dimensional projection. The effect can be demonstrated by using a piece of string on the surface of a globe and pulled tight between the departure and destination cities. Thus, it will be apparent that there are many different routes from London to New York which are of different lengths depending on the route taken. This can be demonstrated again with the piece of string on the globe where the string is deflected sideways from its shortest path, although the path can still be a smooth curve and must be in contact with the surface of the globe for its complete length. This path could represent the shortest flown distance, taking into account winds of different strengths dependent on the path flow. It is entirely possible to pick a relatively long flight path from London to New York which is of equal length to the shortest flight path from London to a destination that is further away, such as Chicago. Similarly, the stent of the present invention can be constructed from filamentary components which have the same length but which follow a different “flight path” along the surface of the stent in order to create a stent of non-uniform cross-section.

In the case of the stent graft, the implant additionally includes flexible material on the outside or inside of the multiple filamentary components which also defines an elongate shape.

Preferably, each of the multiple filamentary components are either formed of an individual filament which terminates at the first and second ends of the structure, or is a part of a longer filament. It will be appreciated that a combination stent can be produced in which a proportion of the filamentary components are individual component and a proportion are formed of the same component which is sufficiently long to follow a path from one end to another of the structure and back again (possibly multiple times). The essential feature is that the length from one end to the other of the structure is substantially identical for all filaments. Thus, said longer filament may have a length which is an integral multiple of the length of said individual filament, and which changes direction at the first end of the structure, the second end of the structure, or both, to form a plurality of said multiple filamentary components (as measured end-to-end).

In accordance with a second aspect of the present invention, there is provided a method for forming a medical implant, including the steps of: providing multiple filamentary components braided into a first elongate structure in which the cross-section of the structure is substantially uniform along its length, and deforming the first elongate structure into a second elongate structure in which the cross-section of the structure is not uniform along its length, wherein each of said multiple filamentary components is substantially equal in length.

A preferred method of achieving this is to design the complex shape initially as a cylinder, prior to a secondary deformation that imparts the complex shape. A second method is to use mandrels with grooves computed to achieve this result. A preferred computation involves lengthening the shortest wires in the braid but maintaining the curved shape of the individual braid wire. The length is extended by deflecting the path of the wire, preferably by means of a smooth curve, from the path of the shortest length. The wire should still be arranged to lie on the surface of the complex shape and not project substantially inwards or outwards from the surface. A third method is effective in some situations. In those situations, the lengths of the filaments are calculated for all starting positions around the circumference of the expanded device. A selection of filaments may be made where the lengths of the filaments are substantially the same and the braid can be made from those selected filaments. It will be appreciated that combinations of these methods can also be used. Thus in a preferred embodiment, the braided stent has a complex shape where a first part of its length is not circular in cross section and where a second part of its length has a different cross sectional shape which may be round and in which every wire filament from the first end to the second end has the same length.

A number of preferred embodiments of the present invention are illustrated in the accompanying drawings, in which:

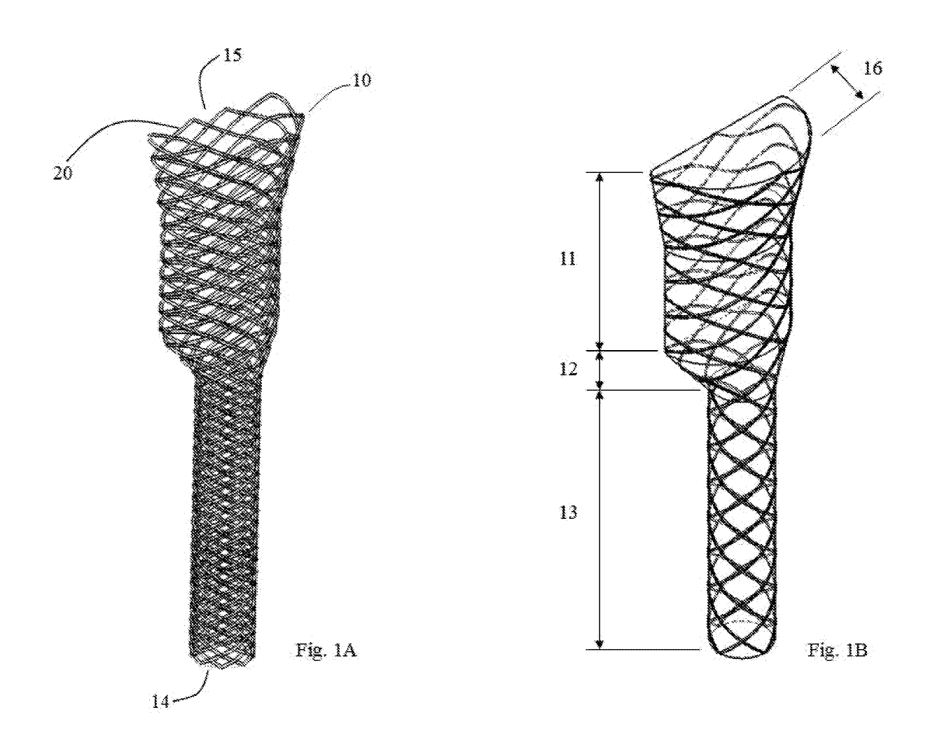

FIG. 1A is a schematic representation of a stent graft in accordance with the invention in its contracted configuration;

FIG. 1B is a schematic representation of a further stent graft in accordance with the invention in its contracted configuration;

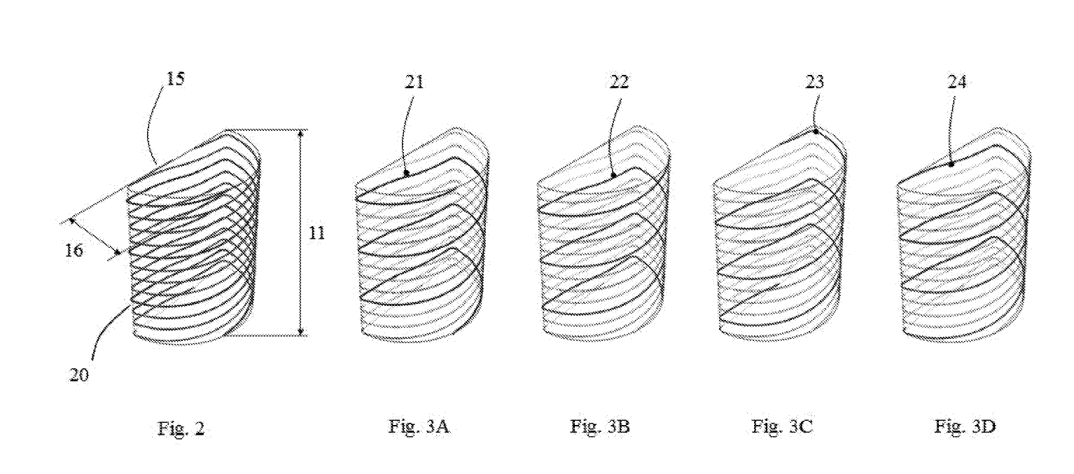

FIG. 2 is a schematic representation of one end of a simplified stent graft in accordance with the invention;

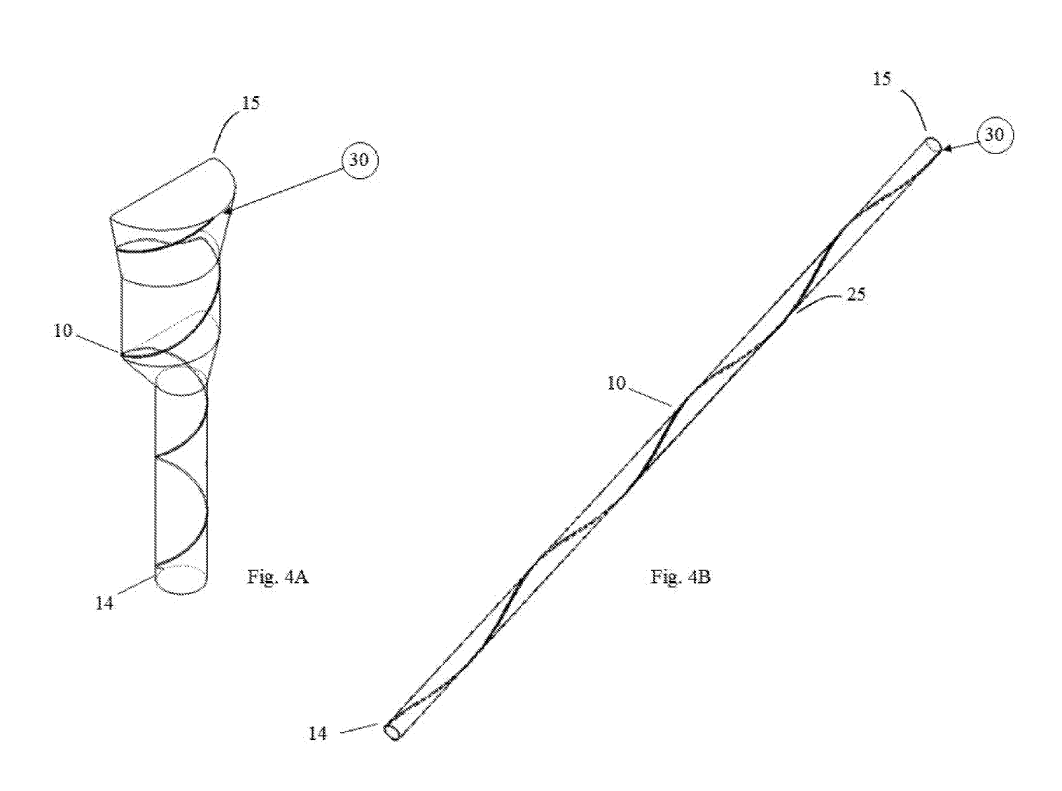

FIGS. 3A to 3D are schematic representations of the arrangement shown in FIG. 2 but with individual wires highlighted in each case;

FIGS. 4A and 4B show a schematic representations of a stent graft in accordance with the present invention in contracted and extended configurations, and

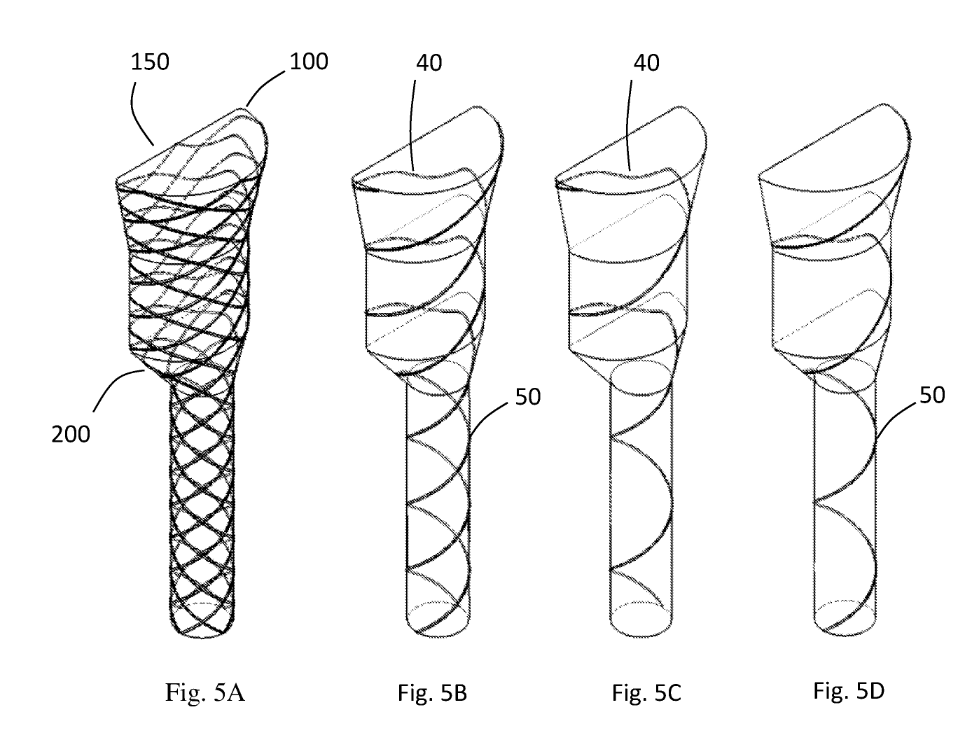

FIGS. 5A to 5D show schematic representations of a stent graft not in accordance with the invention.

Turning to FIGS. 1A and 1B, a stent graft has a braided stent frame 10 formed from a multiplicity wires 20 attached to graft material (not shown). Stent frame 10 is shown in its contracted configuration with ‘D’-shaped longitudinal section 11 (which is 44.4 mm long), transitional section 12 (which is 11 mm long) and circular longitudinal section 13 (which is 60.6 mm long). Thus stent frame 10 is 116 mm long when measured from circular end 14 to D-shaped end 15. The radius 16 of D-shaped end 15 is 18.5 mm and the diameter of circular end 14 is 12.5 mm. It will be appreciated that the pattern of wires 20 is different in the stent frames 10 of FIGS. 1A and 1B but in each case each of the wires 20 has the same length. In the case of stent frame 10 of FIG. 1A the length of each wire is 260 mm and in the case of FIG. 1B it is 260 mm.

D-shaped section 11 of an alternative stent frame 10 is shown in FIG. 2. D-shaped section 11 has length 40 mm and radius 16 of 18.5 mm. Individual wires forming the multiplicity of wires 20 are shown as wires 21, 22, 23 and 24 in FIGS. 3A-3D and are all of equal length (284.3 mm).

Turning to FIG. 4A, this shows a stent frame 10 having graft material 25 thereon in contracted configuration with circular end 14 and D-shaped end 15. The multiplicity of wires have been omitted for clarity except wire 30, which is shown winding in a generally helical form from one end 14 of the stent graft to the other 15.

The stent graft of FIG. 4A is then shown in FIG. 4A in its extended configuration in which it is generally tubular in form with a uniform cross-section which is longer and thinner that the contracted configuration. Wire 30 is also depicted on the stent graft of FIG. 4B, and it can be seen that it retains its generally helical form, but with a more open ‘thread’ than in the contracted configuration.

Because of the nature of the braiding, the stent graft of FIG. 4B can easily be generated by pulling the stent graft of FIG. 4A at either end to stretch it into the extended configuration. If the stent graft of FIG. 4B is released, it pops back into the configuration of FIG. 4A, because of the shape memory of the wires 20.

Finally, FIG. 5A shows a braided stent frame 100 having a multiplicity of wires 200 attached to graft material (not shown). Stent frame 100 is in contracted configuration has circular end 140 and D-shaped end 150 as with stent frame 10 shown in earlier Figures. FIG. 5B is a schematic representation focusing on two specific wires from multiplicity 200, namely wire 40 which is 233.4 mm long and wire 50 which is 226.2 mm long. FIGS. 5C and 5D show individually wire 40 and wire 50 respectively. The fact that the wires forming multiplicity 200 are not of equal length from end-to-end along stent frame 100 is found to cause problems when stent frame 100 is transformed from the contracted configuration shown to the extended configuration (not shown) because the wires 200 are liable to kink or snag, in contrast to the wires forming the stent of the present invention.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosures in UK patent application number 1517813.0, from which this application claims priority, and in the abstract accompanying this application are incorporated herein by reference.

1. A medical implant for implanting in a body lumen, including
multiple filamentary components braided into an elongate structure having a first end and a second end and defining an implant lumen,
wherein the cross-section of the elongate structure is non-uniform along its length or wherein the cross-section of the elongate structure is uniform along its length but non-circular,
and wherein each filamentary component is substantially equal in length as measured from the first end to the second end of the structure.

2. An implant as claimed in claim 1 having a first longitudinal section in which the elongate structure is not circular in cross-section and a second longitudinal section in which the elongate structure is circular in cross-section.

3. An implant as claimed in claim 2, wherein the first longitudinal section has a ‘D’-shaped cross-section.

4. An implant as claimed in claim 1, additionally including flexible material on the outside or inside of the multiple filamentary components which also defines an elongate shape.

5. An implant as claimed in claim 1, wherein each of the multiple filamentary components are either formed of an individual filament which terminates at the first and second ends of the structure, or is a part of a longer filament.

6. An implant as claimed in claim 5, wherein said longer filament has a length which is an integral multiple of the length of said individual filament, and which changes direction at the first end of the structure, the second end of the structure, or both, to form a plurality of said multiple filamentary components.

7. An implant as claimed in claim 1, wherein the multiple filamentary components are part of a single filament which changes direction at the first end and the second of the structure multiple times.

8. A method for forming a medical implant, including the steps of:
providing multiple filamentary components braided into a first elongate structure in which the cross-section of the structure is substantially uniform along its length, and
deforming the first elongate structure into a second elongate structure in which the cross-section of the structure is not uniform along its length,
wherein each of said multiple filamentary components is substantially equal in length.

9. A method as claimed in claim 8, wherein the filamentary components are formed of a shape memory material, and wherein the filamentary components are trained to remember the shape of the second elongate structure.

10. A method as claimed in claim 8, wherein second elongate structure has a first longitudinal section in which the elongate structure is not circular in cross-section and a second longitudinal section in which the elongate structure is circular in cross-section.

11. A method as claimed in claim 10, wherein the first longitudinal section has a ‘D’-shaped cross-section.

12. A method as claimed in claim 8, additionally including the step of providing flexible material on the outside or inside of the filamentary components which is also in the form of an elongate structure.

13. A method as claimed in claim 8, wherein the medical implant that is formed is a medical implant as claimed in claim 1.

14. (canceled)

15. A method for forming a medical implant as claimed in claim 1, including the steps of:
providing a mandrel having a plurality of grooves therein, wherein the grooves are disposed to correspond to the position of filamentary components in an implant as claimed in claim 1, and
placing filaments in said grooves in order to form said implant.

16. A method as claimed in claim 15, wherein the mandrel is used to define the shape of the filaments by plastic deformation, annealing, heat setting or chemical setting.
